(19) United States
(12) Patent Application Publication
(10) Pub. No.: US 2022/0108778 A1
(43) Pub. Date: Apr. 7, 2022

(54) Algorithmic Framework For Customizing Therapeutic or Cosmetic Formulations

(71) Applicant: Robin James Evans, Thousand Oaks, CA (US)

(72) Inventor: Robin James Evans, Thousand Oaks, CA (US)

(21) Appl. No.: 17/495,865

(22) Filed: Oct. 7, 2021

Related U.S. Application Data
(60) Provisional application No. 63/088,785, filed on Oct. 7, 2020.

(51) Int. Cl. G16H 10/40; G16H 10/60; G16H 10/20; G16H 50/20; G16H 50/30

(52) U.S. Cl. CPC G16H 10/40; G16H 10/60; G16H 50/30; G16H 50/20; G16H 10/20

(57) ABSTRACT

A framework for developing a customized therapeutic or cosmetic formulation, and application thereof, that is a specific to an individual, and to the skin condition individual suffers from, for treatment thereof. This framework incorporates input data that together represents clinical data, genomic data, and ingredient data, and applies this information to a neural network to improve simulations of treatments by associating variables extracted from relevant data points relative to the patient and skin condition, and integrating comparisons with both in vivo measurements and conditions experienced by and treated for in similar patients over time. This application of artificial intelligence may be utilized in any medical setting, is not limited to treating dermatological or skin-related ailments.

This application claims the benefit of priority and is entitled to the filing date pursuant to 35 U.S.C. § 119(e) of U.S. Provisional Patent Application 63/088,785, filed Oct. 7, 2021, the content of which is hereby incorporated by reference in its entirety.

### FIELD

The present invention relates to the field of skin science. Specifically, the present invention relates to evaluating clinical data, genomic data and ingredient data to develop a custom therapeutic or cosmetic formulation specific to an individual as a treatment for a skin condition from which the individual is suffering.

### BACKGROUND

There are many existing approaches to treating skin ailments in a dermatological or other medical setting. These existing approaches are often limited, however, to treatment based just on knowledge of the patient's oral description of the problem, a visual examination of the patient by a medical professional, and any biopsies, swabs, or other clinical tests performed. These approaches do not account for a deeper understanding of the patient's genomic makeup influencing the patient's condition that would improve conclusions as to how to diagnose or treat the problem. Additionally, there is no existing approach in the available art that incorporates techniques of machine learning and artificial intelligence to improve upon evaluations of clinical data for developing formulations of available ingredients (and the elements thereof).

Accordingly there is a need in the existing art for an approach that analyzes a patient's genetic makeup to improve treatments of skin conditions that are unique to the individual. There is also a need for an approach that models possible customized therapeutic or cosmetic formulations using such techniques of machine learning and artificial intelligence for treating a patient's skin condition. There is also a need for approach that develops an application schedule for treating a patient's skin condition using such customized therapeutic or cosmetic formulations.

### SUMMARY

The present invention addresses these issues by providing a proprietary algorithmic framework for modeling various types of information to arrive at a custom formula and application thereof that is specific to an individual for treating a skin condition. This proprietary algorithmic framework takes multiple variables into account, including a thorough clinical assessment, in vivo measurements, and a patient's genomic structure, as well as available ingredients of treatment options. Such an algorithmic framework may be applied for any medical condition, and therefore has utility beyond dermatological or skin-related ailments.

It is therefore one objective of the present invention to provide a system and method of evaluating an individual. It is another objective of the present invention to provide a system and method of evaluating an individual and generating a customized therapeutic or cosmetic treatment for that particular individual. It is a further objective of the present invention to provide a system and method of evaluating an individual that includes an assessment of clinical data and genomic data for the individual, and ingredient information for developing the customized therapeutic or cosmetic treatment for that particular individual. It is still another objective of the present invention to provide a system and method that applies one or more techniques of machine learning and artificial intelligence to develop customized therapeutic or cosmetic formulations for a particular individual.

The present invention further provide a system and method of evaluating a patient's skin condition. It is another objective of the present invention to provide a system and method of evaluating a patient's skin condition and generating a customized therapeutic or cosmetic treatment for a particular patient. It is a further objective of the present invention to provide a system and method of evaluating a patient's skin condition that includes an assessment of clinical data, genomic data, and ingredient information for developing the customized therapeutic or cosmetic treatment. It is still another objective of the present invention to provide a system and method that applies one or more techniques of machine learning and artificial intelligence to develop customized therapeutic or cosmetic formulations for treating dermatological conditions.

Other objects, embodiments, features and advantages of the present invention will become apparent from the following description of the embodiments, taken together with the accompanying drawings, which illustrate, by way of example, the principles of the invention.

### BRIEF DESCRIPTION OF THE DRAWINGS

The accompanying drawings, which are incorporated in and constitute a part of this specification, illustrate aspects of the disclosed subject matter in at least one of its exemplary embodiments, which are further defined in detail in the following description. Features, elements, and aspects of the disclosure are referenced by numerals with like numerals in different drawings representing the same, equivalent, or similar features, elements, or aspects, in accordance with one or more embodiments. The drawings are not necessarily to scale, emphasis instead being placed upon illustrating the principles herein described and provided by exemplary embodiments of the invention. In such drawings:

FIG. 1 is a system architecture diagram illustrating functional components in an algorithmic framework for developing a customized therapeutic or cosmetic formula, for treating skin conditions, according to one embodiment of the present invention;

FIG. 2 is a system architecture diagram illustrating functional components in an algorithmic framework for compiling clinical assessment data, according to one embodiment of the present invention;

FIG. 3 is a system architecture diagram illustrating functional components in an algorithmic framework for compiling genomic data, according to one embodiment of the present invention; and

FIG. 4 is a system architecture diagram illustrating functional components in an algorithmic framework for compiling ingredient data, according to one embodiment of the present invention.

### DETAILED DESCRIPTION

In the following description of the present invention, reference is made to the exemplary embodiments illustrating the principles of the present invention and how it is practiced. Other embodiments will be utilized to practice the present invention and structural and functional changes will be made thereto without departing from the scope of the present invention.

The present invention is an algorithmic framework for developing a custom therapeutic or cosmetic formulation and application thereof, that is a specific to an individual and to the skin condition for which the individual is being treated. This framework incorporates input data that together represents clinical data, genomic data, and ingredient data, and incorporates techniques of machine learning and artificial intelligence to train a neural network to improve analysis and development of customized therapeutic and cosmetic formulations. This may occur, for example by associating variables extracted from relevant data points among the various types of input data, with those relative to comparisons with both in vivo measurements and conditions experienced by and treated for in similar patients over time.

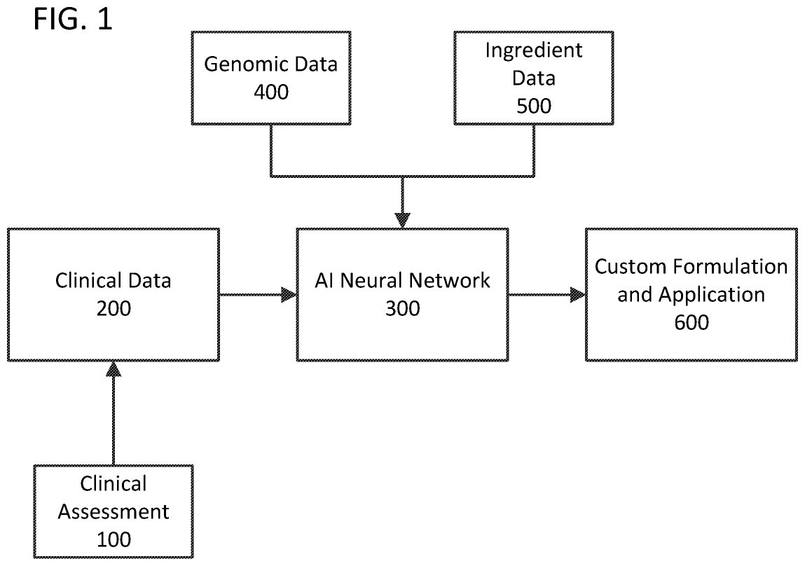

FIG. 1 is a schematic diagram of the algorithmic framework of the present invention. In this framework, and as noted above, input data in the form of clinical data 200, genomic data 400, and ingredient data 500 are applied to a neural network 300 to generate a customized therapeutic or cosmetic formulation and application thereof 600. Each element of the input data includes many types of information which are evaluated in this algorithmic framework to develop the customized therapeutic or cosmetic formulation and application thereof for specific patients and their conditions.

The algorithmic framework is performed in conjunction with, and operates within, a computing environment in which one or more processors and a plurality of software and hardware components may be configured to execute program instructions or routines to perform the elements and data processing functions described herein and embodied in one or more algorithms. These algorithms are part of a proprietary environment in which one or more systems and methods are performed by applying mathematical functions, models or other analytical and data processing techniques that ensure that a custom formula, and an application thereof that is a specific to an individual, and to the skin condition for which the individual is being treated, is developed and generated.

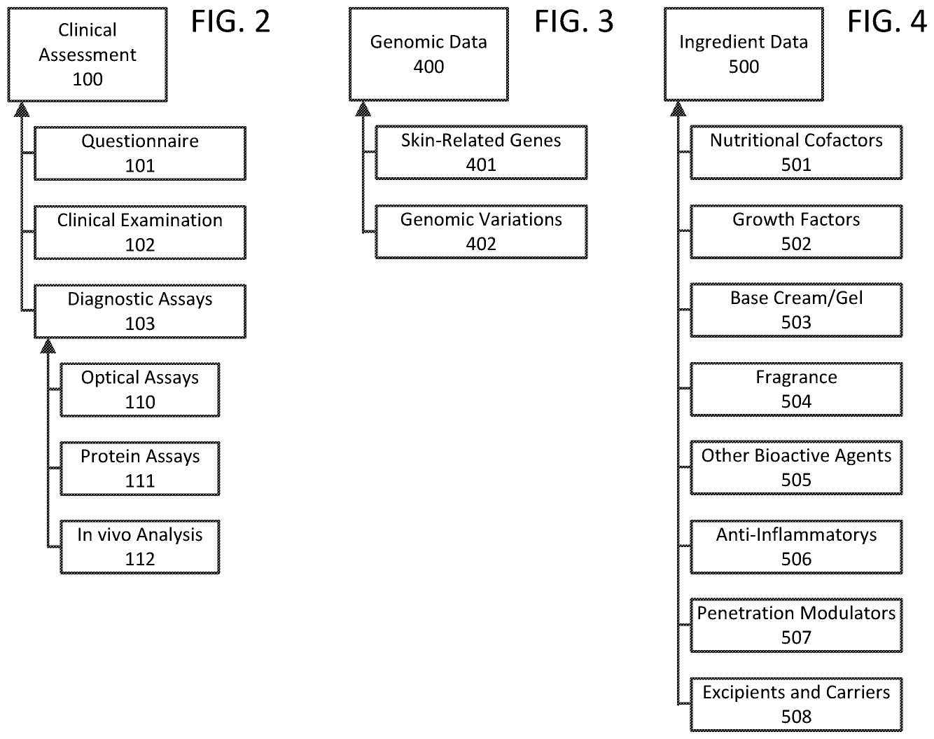

As shown in FIG. 1, clinical data 200 includes a clinical assessment 100 of an individual. As shown in FIG. 2, in the algorithmic framework of the present invention clinical assessment 100 of individual can begin with a standardized questionnaire 101 that informs the clinician as to the general skin subtype. This standardized questionnaire 101 may be performed in a clinical setting in person, or electronically, and the questionnaire may involve written answers from a patient or may be administered orally by the clinician.

Referring to FIG. 2, following standardized questionnaire 101, the clinical assessment 100 may continue with a clinical examination 102 with a skin care specialist. This specialist reviews and logs the patient's history and current skin care products being used. Clinical examination 102 allows for broad categorization and quantification of an individual's overall skin condition into different skin types and problems. For example, collagen quality, skin elasticity, fine lines and wrinkles, acne, loss of volume, sun damage, pigmentation irregularities, and inflammation can be qualitatively assessed. General skin condition can be categorized as being oily, dry, inflammatory, aging, pigmentation and/or acne with. Any given patient can have a combination of these conditions, and they may be represented by percentages in the clinical examination 102. Thus, the objective of clinical examination 102 is to quantify the patient's overall skin condition and concerns into percentages of available skin categories as noted above.

As shown in FIG. 2, clinical assessment 100 may also include diagnostic and other assays 103 to further supplement the patient's overall skin condition and provide depth, substance and improve the quality of the clinical assessment 100. Diagnostic and other assays 103 include, e.g., optical assays 110, like fluoroscopy and spectroscopy tests, protein assays 111, and in vivo data analysis 112.

Optical assays 110 utilize optical technology to provide a quantitative measure of a patient's skin composition, such as fluoroscopy/spectroscopy tests. Fluorescence occurs when light is absorbed at one wavelength and emitted at a higher one. The absorption of photons occurs with different chromophores in the skin, and fluorophores are molecules that emit light in response to light excitation. One type of optical assay 110 is fluorescence spectroscopy, which allows for quantification of biochemical and morphological properties of skin, which allows for outcome measurement before and after treatment. Fluorophores are excited predominately in the ultraviolet (UV) range. Endogenous fluorophores include, but are not limited to, aromatic amino acids, protoporphyrins, collagen/elastin, NAD(P)H, fatty acids, vitamin A, flavins, and lipofuscins. Regardless, each of these fluorophores are quantified by an intensity and location in fluorescence spectroscopy, and this allows for creation of a topical map for quantification of skin qualities in a particular patient.

Another optical assay 110 is Raman spectroscopy, which is a relatively newer technique for skin quantification of a patient. The Raman effect occurs when energy is exchanged between light and matter. When light strikes a substance, it is either scattered or absorbed. Most of the scattered light will have the same frequency and wavelength as the incident photons. However, a small fraction will be scattered in a different energy form (usually lower) than the incident photons, and these are termed Raman scattered photons. This allows for improved resolution and a wider variety of molecules that can be quantified.

A typical source of excitation for Raman spectroscopy is a Nd:YAG laser. For instance, quantification of lipids, proteins and water in the skin becomes possible in vivo using such a laser. The data collected in Raman spectroscopy is presented as Raman spectra. A Fourier transform is typically applied to such data, and peaks are identified from a sample. This can be correlated to standardized locations on the face and body, which can be utilized for comparison purposes. Data collected from the fluoroscopy/spectroscopy tests is represented by means of 2D facial maps, which allows for differential information to be developed.

Diagnostic and other assays 103 may also include a protein assay 111. The direct measurement of proteins allows for measurement of expression. A protein assay 111 is a non-invasive test that is especially useful in the measurement of cytokines as a marker of skin inflammation. Use of tapes or other superficial collection methods allows for quantification of such a test. Protein assays 111 also allow for quantification of skin microbiome.

Diagnostic and other assays 103 can also integrate data from in vivo measurements regarding the skin. Such in vivo analysis 112 may include a sequence of topographical maps with intensity values to distinguish severity of several different skin characteristics.

Clinical data 200 collected as a result of the clinical assessment 100 summarized by combining the overall percentages for each category obtained from the questionnaire 101, the clinical examination 102, and diagnostic and other assays 103. This allows for development of multiple qualitative variables, also represented as percentages.

As noted in FIG. 1, another component of input data in the algorithmic framework is a patient's genomic data 400. Referring to FIG. 3, genomic data 400 includes information about skin-related genes 401, and genomic variations 402. The identification of skin-related genes 401 in a patient allow for quantification of the importance of clinical data, treatment selection, and prediction of future skin outcomes.

Skin aging is related to both intrinsic and extrinsic factors, and the interaction of these factors. Intrinsic skin aging accounts for approximately 30% of aging and is directly related to a person's genetic makeup. Each person's intrinsic aging process can be accelerated by environmental factors, such as for example exposure to the sun. Additionally, collagen, skin elasticity, advanced glycation end products, pigmentation, antioxidants, inflammation, hydration, and other genetic-related disorders are all important variables in overall skin quality and the effects of both intrinsic and extrinsic aging.

Collagen is the main structural protein in skin, and accounts for the development of wrinkles and fine lines. Collagen is found in the dermis, and is comprised of triple-helixes of fibrils. Type 1 collage is found within the skin, and is comprised of two pro-a1 chains and one pro-a2 chain. The pro-a1 fibrils are coded by COL1A1 and the pro-a2 by COL1A2, respectively. Known genetic mutations in COL1A1 and COL1A2 are informative for a patient's genomic data, as they can cause Ehlers-Danlos syndrome, osteogenesis imperfectica, carpal tunnel syndrome, dermatofibrosarcoma protuberans, scleroderma, and degenerative disk disease. Also, subtle differences in collagen formation and metabolism can have drastic effects on aging. Aging skin is, in fact, marked by increased cross-linking and a rigid quality. Other known related collagen genes include TNF, IL6, FN1, ELN, IGF1 and 4899 other genes.

Another genomic indicator is skin elasticity, which is predominately regulated by elastin. Young, healthy skin has a large percentage of elastin to allow for a tight and responsive contour. Soluble proteins known as tropoelastin are linked together to form elastin. This protein contains alternative hydrophobic and hydrophilic domains. The collagen and elastin ratio is a well known indicator of skin youthfulness.

As noted in FIG. 1, another component of input data in the algorithmic framework is a ingredient data 500 taken from the ingredients used to make the customized therapeutic or cosmetic formulation. Referring to FIG. 4, ingredient data 500 comprise many types of specific information including nutritional cofactors 501 and growth factors 502, as well as product specific attributes such as the type of base cream/gel 503 and fragrance 504. Other components of ingredient 500 used in the algorithmic framework include bioactive molecules and extracts 505 present in an ingredient, anti-inflammatory factors 506, and penetration modulators 507 as well as excipients and carriers 508. Together, these elements represent available ingredients for treating a particular skin condition that the algorithmic framework may generate as part of a customized therapeutic or cosmetic formulation for treating a patient's skin condition.

Each of these specific aspects of ingredients may play a role in treating (or worsening) a patient's skin condition, informed by the clinical data 200 and the genomic data 400. For example, an inflammatory control element of a therapeutic or cosmetic formulation may be very important to treating a particular skin condition, while a presence of a chemical inducing a particular fragrance (or other additive) may worsen an existing condition. Therefore, the neural network 300 of the present invention is designed to evaluate the many different ingredients of available treatments to arrive at the most appropriate solution for a patient.

As noted in FIG. 1, each element of the input data is applied to a neural network 300. The neural network 300 is an application of artificial intelligence to the algorithmic framework of the present invention, in which a set of training data includes data from in vivo measurements as noted above to analyze clinical data, the associated optical scans, and genomic data, in a three-dimensional analysis. The application of artificial intelligence in the present invention therefore applies the neural network(s) to improve correlations between the various types of input data to generate customized treatment formulations and applications thereof for a patient's specific skin condition.

This unique application of artificial intelligence enhances the dataset of clinical information and improves the understanding of detected skin conditions that have developed, and also informs the overall personalized diagnosis. Also, the learning algorithm of this neural network 300 is improved by optimization both within the patient data set, and by comparison to similar patients over time.

The neural network 300 represents an application of artificial intelligence to associate and compare variables in the various types of input data and identify relationships in such input data to generate a custom therapeutic or cosmetic formulation specific to an individual, and a treatment using such formulation for the skin condition from which the individual is suffering. The algorithmic framework of the present invention contemplates that the relationships among the various types of input data may be identified and developed by training the neural network to continually analyze input data, to build a more comprehensive dataset that can be used to make improvements to the outputs represented by the custom therapeutic or cosmetic formulation, and a corresponding treatment using such formulation.

For instance, the application of artificial intelligence in the algorithmic framework can be applied to an adequately sized dataset to draw automatic associations, correlations and identify relationships between the available input data from clinical data 200, genomic data 400 and ingredient data 500, effectively yielding a customized neural network for simulating types of formulations and applications thereof to treat skin conditions. As more and more data are accumulated, the information can be sub-sampled, the neural network 300 retrained, and the results tested against independent data to further customize the resultant formulations and treatments. Further, this may yield information as to the importance of related factors through weighting of variables between inputs, and may be further used to identify which factors would be particularly important or unimportant in a treatment, and thus help to target ways of improving the neural network over time.

The algorithmic framework of the present invention contemplates that many different types of artificial intelligence may be employed and are within the scope thereof. The application of artificial intelligence may include, in addition or lieu of the neural network 300, one or more of such types of artificial intelligence. These may include, but are not limited to, techniques such as k-nearest neighbor (KNN), logistic regression, support vector machines or networks (SVM), and instantiations of one or more other types of machine learning paradigms such as supervised learning, unsupervised learning and reinforcement learning. Regardless, the use of artificial intelligence in the algorithmic framework of the present invention enhances the utility of data processing functions performed therein by automatically and heuristically constructing appropriate relationships, mathematical or otherwise, relative to the complex interactions between data obtained from a clinical assessment of a patient, the patient's genomic information, and available compounds for treating a particular skin condition, to arrive at the most appropriate formulation and application thereof for the patient. For example, where predictive factors known to be related to a particular skin condition are known and measured, along with the actual outcomes in in vivo measurements, machine learning techniques are used to ‘train’ or construct a neural network 300 that relates the more readily available predictors to the ultimate outcomes.

There are three algorithms running in parallel. Firstly, there is the creation of a unique patient profile. This is derived from the correlation of data between the genomic, proteomic, clinical and optical assays. The relationship between this data is noted between participants. Multilinear regression techniques allow for the development of a model that interprets both the quantitative and qualitative data. This allows for the development of potentially new skin classifications based on empirical data. The development of this profile is constantly improved with the addition of new patient data.

The second algorithm, running in parallel, is the formulation creation machine learning algorithm. Given a particular profile as disclosed above using the first algorithm, the likely effective therapeutic compounds will be evaluated and ranked. This second algorithm will weight the effectiveness weighting of the effectiveness of the therapeutic compounds in order to determine the concentration and dose of the therapeutic compounds as well as carrier and excipients to be used in the formulation. This formulation will change according to time-series feedback created by ongoing changes in monitored data such as proteomics, optical assays and clinical data.

The final algorithm relies on a molecular database of therapeutic compounds to assist in the development of new compounds. Development of a real-time chemoinformatic database assist in identifying potential new compounds and improving the weighting of existing compounds in the formulation algorithm in a particular pharmacogenetic profile. This database is run in parallel with the profile and formulation creation algorithms. The database detail includes but is not limited to molecular weight, molecular structure, potency, selectivity, absorption, metabolism, and toxicity. The use of structure linear notation, pre-defined substructures and other characteristics will be included in our compound library. Multidimensional scaling (MDS) and principal component analysis (PCA) may be utilized to visualize important pharmacogenetic characteristics of both existing and to predict potentially new therapeutic compounds.

The three algorithms (profile algorithm, formulation algorithm and new compounds development algorithm) run in parallel but do have feedback to each other to achieve an optimized outcome. The weighting of the compounds within the formulation algorithm are optimized according to the regression algorithm based on ongoing optical assay, proteins assay and clinical information in the context of the profile algorithm. The effectiveness of a given formulation is correlated to optimize the subcategories of the profile algorithm. Finally, the new compound algorithm suggests new therapeutics for introduction for a particular profile in the context of a particular formulation. The incorporation of new therapeutics into the formulation algorithm can be automated, semi-supervised or completely separate.

The systems and methods for performing the algorithmic framework of the present invention may be implemented in many different computing environments. For example, the framework for hosting and executing subscription-based, end-to-end agricultural workflows in a permissioned, distributed ledger may be implemented in conjunction with a special purpose computer, a programmed microprocessor or microcontroller and peripheral integrated circuit element(s), an ASIC or other integrated circuit, a digital signal processor, electronic or logic circuitry such as discrete element circuit, a programmable logic device or gate array such as a PLD, PLA, FPGA, PAL, and any comparable means. In general, any means of implementing the methodology illustrated herein can be used to implement the various aspects of the present invention. Exemplary hardware that can be used for the present invention includes computers, handheld devices, telephones (e.g., cellular, Internet enabled, digital, analog, hybrids, and others), and other such hardware. Some of these devices include processors (e.g., a single or multiple microprocessors), memory, nonvolatile storage, input devices, and output devices. Furthermore, alternative software implementations including, but not limited to, distributed processing, parallel processing, or virtual machine processing can also be configured to perform the methods described herein.

The systems and methods of the present invention may also be partially implemented in software that can be stored on a storage medium, executed on programmed general-purpose computer with the cooperation of a controller and memory, a special purpose computer, a microprocessor, or the like. In these instances, the systems and methods of this invention can be implemented as a program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Additionally, the data processing functions disclosed herein may be performed by one or more program instructions stored in or executed by such memory, and further may be performed by one or more modules configured to carry out those program instructions. Modules are intended to refer to any known or later developed hardware, software, firmware, artificial intelligence, fuzzy logic, expert system or combination of hardware and software that is capable of performing the data processing functionality described herein.

In closing, foregoing descriptions of embodiments of the present invention have been presented for the purposes of illustration and description. It is to be understood that, although aspects of the present invention are highlighted by referring to specific embodiments, one skilled in the art will readily appreciate that these described embodiments are only illustrative of the principles comprising the present invention. As such, the specific embodiments are not intended to be exhaustive or to limit the invention to the precise forms disclosed. Therefore, it should be understood that embodiments of the disclosed subject matter are in no way limited to a particular element, compound, composition, component, article, apparatus, methodology, use, protocol, step, and/or limitation described herein, unless expressly stated as such.

In addition, groupings of alternative embodiments, elements, steps and/or limitations of the present invention are not to be construed as limitations. Each such grouping may be referred to and claimed individually or in any combination with other groupings disclosed herein. It is anticipated that one or more alternative embodiments, elements, steps and/or limitations of a grouping may be included in, or deleted from, the grouping for reasons of convenience and/or patentability. When any such inclusion or deletion occurs, the specification is deemed to contain the grouping as modified, thus fulfilling the written description of all Markush groups used in the appended claims.

Furthermore, those of ordinary skill in the art will recognize that certain changes, modifications, permutations, alterations, additions, subtractions and sub-combinations thereof can be made in accordance with the teachings herein without departing from the spirit of the present invention. Furthermore, it is intended that the following appended claims and claims hereafter introduced are interpreted to include all such changes, modifications, permutations, alterations, additions, subtractions and sub-combinations as are within their true spirit and scope. Accordingly, the scope of the present invention is not to be limited to that precisely as shown and described by this specification.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

The words, language, and terminology used in this specification is for the purpose of describing particular embodiments, elements, steps and/or limitations only and is not intended to limit the scope of the present invention, which is defined solely by the claims. In addition, such words, language, and terminology are to be understood not only in the sense of their commonly defined meanings, but to include by special definition in this specification structure, material or acts beyond the scope of the commonly defined meanings. Thus, if an element, step or limitation can be understood in the context of this specification as including more than one meaning, then its use in a claim must be understood as being generic to all possible meanings supported by the specification and by the word itself.

The definitions and meanings of the elements, steps or limitations recited in a claim set forth below are, therefore, defined in this specification to include not only the combination of elements, steps or limitations which are literally set forth, but all equivalent structure, material or acts for performing substantially the same function in substantially the same way to obtain substantially the same result. In this sense it is therefore contemplated that an equivalent substitution of two or more elements, steps and/or limitations may be made for any one of the elements, steps or limitations in a claim set forth below or that a single element, step or limitation may be substituted for two or more elements, steps and/or limitations in such a claim. Although elements, steps or limitations may be described above as acting in certain combinations and even initially claimed as such, it is to be expressly understood that one or more elements, steps and/or limitations from a claimed combination can in some cases be excised from the combination and that the claimed combination may be directed to a sub-combination or variation of a sub-combination. As such, notwithstanding the fact that the elements, steps and/or limitations of a claim are set forth below in a certain combination, it must be expressly understood that the invention includes other combinations of fewer, more or different elements, steps and/or limitations, which are disclosed in above combination even when not initially claimed in such combinations. Furthermore, insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Accordingly, the claims are thus to be understood to include what is specifically illustrated and described above, what is conceptually equivalent, what can be obviously substituted and also what essentially incorporates the essential idea of the invention.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as, e.g., “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising”, variations thereof such as, e.g., “comprise” and “comprises”, and equivalent open-ended transitional phrases thereof like “including”, “containing” and “having”, encompass all the expressly recited elements, limitations, steps, integers, and/or features alone or in combination with unrecited subject matter; the named elements, limitations, steps, integers, and/or features are essential, but other unnamed elements, limitations, steps, integers, and/or features may be added and still form a construct within the scope of the claim. Specific embodiments disclosed herein may be further limited in the claims using the closed-ended transitional phrases “consisting of” or “consisting essentially of” (or variations thereof such as, e.g., “consist of”, “consists of”, “consist essentially of”, and “consists essentially of”) in lieu of or as an amendment for “comprising.” When used in the claims, whether as filed or added per amendment, the closed-ended transitional phrase “consisting of” excludes any element, limitation, step, integer, or feature not expressly recited in the claims. The closed-ended transitional phrase “consisting essentially of” limits the scope of a claim to the expressly recited elements, limitations, steps, integers, and/or features and any other elements, limitations, steps, integers, and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps, integers, and/or features specifically recited in the claim, whereas the meaning of the closed-ended transitional phrase “consisting essentially of” is being defined as only including those elements, limitations, steps, integers, and/or features specifically recited in the claim and those elements, limitations, steps, integers, and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such, the embodiments described herein or so claimed with the phrase “comprising” expressly and unambiguously provide description, enablement, and support for the phrases “consisting essentially of” and “consisting of.”

Lastly, all patents, patent publications, and other references cited and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard is or should be construed as an admission that the inventors are not entitled to antedate such disclosure by virtue of prior invention or for any other reason. All statements as to the date or representation as to the contents of these documents are based on the information available to the applicant and do not constitute any admission as to the correctness of the dates or contents of these documents.

1. A method of identifying an appropriate treatment for a skin condition, comprising:
performing a clinical assessment of a patient, the clinical assessment including interviewing the patient, obtaining information from one or more optical imaging tests that include fluorescence spectroscopy and Raman spectroscopy, obtaining information from a protein assay, and collecting one or more in vivo measurements of the patient's skin;
identifying genomic data of the patient that includes a presence of one or more genes related to particular skin conditions, and genomic variations of the one or more genes related to particular skin conditions, the genomic data at least indicative of variables affecting an intrinsic aging of the patient's skin;
identifying one or more ingredients for formulas for treating skin conditions;
applying the clinical assessment, the genomic data, and the one or more ingredients to a neural network configured to develop correlations between variables affecting a treatment of the skin condition, and perform a multi-dimensional evaluation of the treatment for the skin condition of the patient; and
generating a list of ingredients of a customized therapeutic or cosmetic formula, and an application schedule for the customized therapeutic or cosmetic formula, for treating the skin condition of the patient.

2. A system for identifying an appropriate treatment for a skin condition, comprising:
a computing environment including at least one non-transitory computer-readable storage medium having program instructions stored therein and a computer processor operable to execute the program instructions to model treatments for treating a particular skin condition of a patient, within
a plurality of data processing modules that include:
a module for gathering clinical information from a patient, configured to ingest data representative of a clinical assessment that includes interviewing the patient, obtaining information from one or more optical imaging tests that include fluorescence spectroscopy and Raman spectroscopy, obtaining information from a protein assay, and collecting one or more in vivo measurements of the patient's skin;
a genomic module configured to identify genomic data of the patient that includes a presence of one or more genes related to particular skin conditions, and genomic variations of the one or more genes related to particular skin conditions, the genomic data at least indicative of variables affecting an intrinsic aging of the patient's skin;
an ingredients database configured to store and maintain one or more ingredients for formulas for treating skin conditions;
an artificial intelligence engine configured to apply the clinical assessment, the genomic data, and the one or more ingredients to one or more neural networks configured to develop correlations between variables affecting a treatment of the skin condition, and perform a multi-dimensional evaluation of the treatment for the skin condition of the patient; and
a module configured to generate a list of ingredients of a customized therapeutic or cosmetic formula, and an application schedule for the customized therapeutic or cosmetic formula, for treating the skin condition of the patient.
